(19) United States
(12) Patent Application Publication
(10) Pub. No.: US 2026/0142004 A1
(43) Pub. Date: May 21, 2026

(54) CLINICAL AI QUALITY, BIAS, AND EQUITY MONITORING GOVERNANCE SYSTEM

(71) Applicant: George William Bickerstaff, III, Greenwich, CT (US)

(72) Inventor: George William Bickerstaff, III, Greenwich, CT (US)

(21) Appl. No.: 19/445,527

(22) Filed: Jan. 10, 2026

(51) Int. Cl. G16H 15/00; G16H 10/60

(52) U.S. Cl. CPC G16H 15/00; G16H 10/60

(57) ABSTRACT

A clinical AI quality, bias, and equity monitoring governance system computes stratified performance metrics for AI-assisted clinical workflows and detects equity signals across defined cohorts. The system supports governance review, mitigation tracking, and regulatory-ready reporting while preserving clinician authority.

### TECHNICAL FIELD

The present invention relates to healthcare analytics and governance systems and, more particularly, to computer-implemented systems and methods for monitoring, measuring, and governing quality, bias, and equity characteristics of artificial intelligence-assisted clinical workflows.

### BACKGROUND

Artificial intelligence systems are increasingly deployed within clinical environments to support prioritization, workflow coordination, and decision support.

Post-deployment performance of such systems may vary across patient populations, care settings, and operational contexts, resulting in unintended disparities.

These disparities may arise from imbalanced training data, workflow differences, infrastructure variation, or feedback loops introduced during operational use.

Existing quality and bias assessments are often retrospective, manually conducted, and poorly integrated with live clinical operations.

Accordingly, there exists a need for a technical governance system that continuously monitors quality, bias, and equity characteristics of AI-assisted clinical workflows in a controlled, auditable, and regulator-safe manner without autonomously diagnosing medical conditions or directing treatment actions.

### SUMMARY OF THE INVENTION

The invention provides a computer-implemented clinical AI quality, bias, and equity monitoring governance system configured to analyze workflow events, outcomes, and contextual attributes associated with AI-assisted care delivery.

The system computes stratified performance metrics, bias indicators, and equity signals across defined cohorts, care settings, and monitoring windows.

The system operates as a technical oversight and analytics layer and does not autonomously diagnose medical conditions or prescribe treatment actions.

All computed metrics, detected disparities, and governance actions are recorded as immutable audit artifacts suitable for institutional oversight and regulatory review.

### Definitions (Alphabetical Order)

Bias Indicator refers to a computed metric reflecting differential system behavior across defined cohorts.

Clinical Outcome Metric refers to a measurable result associated with a governed clinical workflow.

Cohort Definition refers to a rule-based grouping of workflow items or patients for analytic comparison.

Equity Signal refers to a detected disparity in quality or performance metrics across cohorts.

Monitoring Window refers to a defined time interval over which system behavior is evaluated.

Performance Drift refers to a change in system metrics relative to a defined baseline.

Quality Threshold refers to a predefined acceptable range for performance metrics.

Stratified metric refers to a metric computed independently for each defined cohort.

Workflow Analytics Engine refers to a software component that computes governance metrics from workflow data.

Workflow Context Attribute refers to metadata describing patient, setting, or operational context associated with a workflow event.

### BRIEF DESCRIPTION OF THE DRAWINGS

FIG. 1 illustrates a clinical AI quality governance architecture.

FIG. 2 illustrates cohort definition and stratified metrics.

FIG. 3 illustrates bias and equity signal detection.

FIG. 4 illustrates governance review and mitigation workflows.

FIG. 5 illustrates audit logging and reporting.

### DETAILED DESCRIPTION

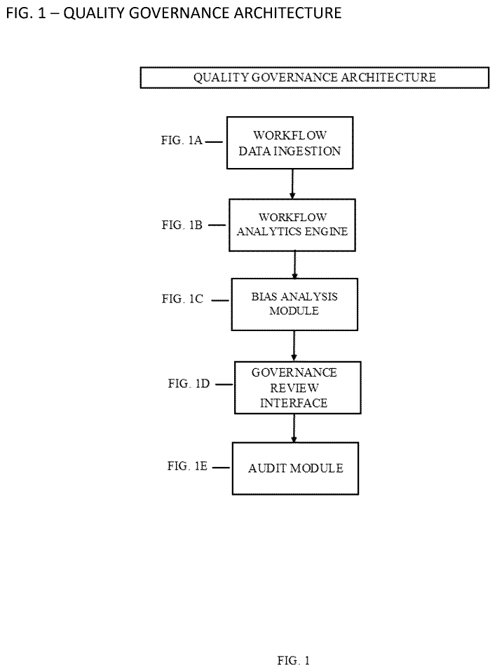

### FIG. 1—Quality Governance Architecture

FIG. 1 illustrates a clinical AI quality, bias, and equity governance system comprising workflow data ingestion, analytics computation, bias detection, governance review, and audit components. The system integrates with AI-assisted workflows without influencing clinician decision authority. Deployment may occur within institutional analytics platforms or secure governance environments.

FIG. 1A—WORKFLOW DATA INGESTION. FIG. 1A depicts ingestion of workflow events and outcomes associated with AI-assisted care. Ingested data includes timestamps and workflow context attributes. Ingestion is passive and non-interventional.

FIG. 1B—WORKFLOW ANALYTICS ENGINE. FIG. 1B illustrates computation of quality and outcome metrics from workflow data. Computation is deterministic and reproducible. Results are stored for downstream analysis.

FIG. 1C—BIAS ANALYSIS MODULE. FIG. 1C depicts evaluation of stratified metrics across cohorts. The module detects disparities and generates bias indicators. Configuration is policy-driven.

FIG. 1D—GOVERNANCE REVIEW INTERFACE. FIG. 1D illustrates presentation of metrics and equity signals to authorized reviewers. The interface is read-only. Access is role-controlled.

FIG. 1E—AUDIT MODULE. FIG. 1E depicts recording of metrics, detections, and governance actions. Records are immutable and time-stamped. Audit artifacts support oversight.

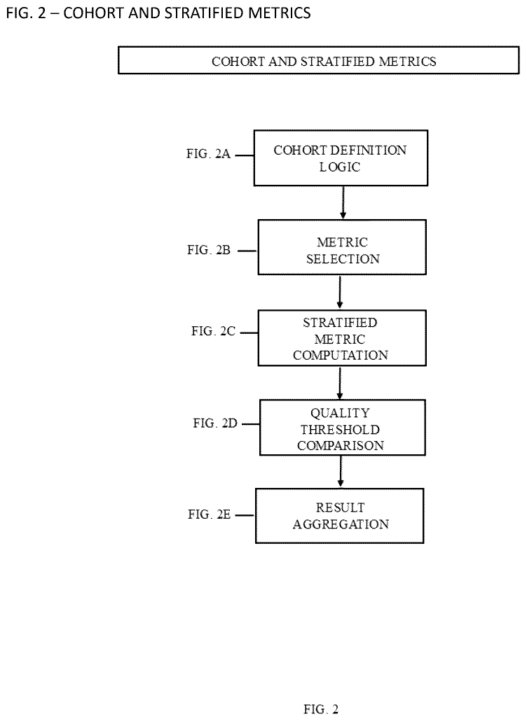

### FIG. 2—Cohort and Stratified Metrics

FIG. 2 illustrates cohort definition and stratified metric computation. Stratification enables equity analysis without affecting workflows. Results support governance decisions.

FIG. 2A—COHORT DEFINITION LOGIC. FIG. 2A depicts cohort definition based on workflow context attributes. Attributes may include demographics or care setting. Definitions are configurable.

FIG. 2B—METRIC SELECTION. FIG. 2B illustrates selection of quality and outcome metrics. Metrics may include response time or escalation rate. Selection is policy-driven.

FIG. 2C—STRATIFIED METRIC COMPUTATION. FIG. 2C depicts computation of metrics independently for each cohort. Computation preserves cohort boundaries. Results are logged.

FIG. 2D—QUALITY THRESHOLD COMPARISON. FIG. 2D illustrates comparison of stratified metrics to quality thresholds. Deviations are identified. Deviations are recorded.

FIG. 2E—RESULT AGGREGATION. FIG. 2E depicts aggregation of stratified results for reporting. Aggregation preserves traceability. Aggregated outputs are auditable.

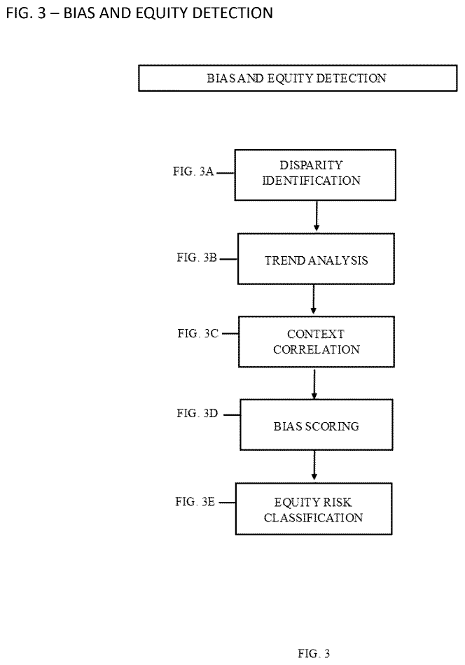

### FIG. 3—Bias and Equity Detection

FIG. 3 illustrates detection of bias indicators and equity signals. Detection uses transparent analytic rules. Detection does not modify workflows.

FIG. 3A—DISPARITY IDENTIFICATION. FIG. 3A depicts identification of statistically significant disparities across cohorts. Disparities are flagged. Flags are logged.

FIG. 3B—TREND ANALYSIS. FIG. 3B illustrates analysis of metrics across monitoring windows. Trends reveal performance drift. Drift is quantified.

FIG. 3C—CONTEXT CORRELATION. FIG. 3C depicts correlation between context attributes and outcomes. Correlation supports analysis. Correlation does not imply causation.

FIG. 3D—BIAS SCORING. FIG. 3D illustrates computation of bias indicator scores. Scores summarize detected disparities. Scores are versioned.

FIG. 3E—EQUITY RISK CLASSIFICATION. FIG. 3E depicts classification of equity risk levels. Classification guides governance review. Rules are configurable.

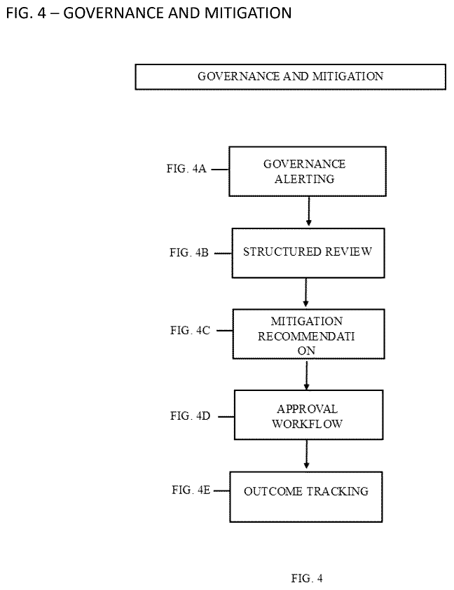

### FIG. 4—Governance and Mitigation

FIG. 4 illustrates governance review and mitigation workflows triggered by equity signals. Workflows support oversight. Workflows do not alter care delivery.

FIG. 4A—GOVERNANCE ALERTING. FIG. 4A depicts alerting of governance stakeholders. Alerts are informational. Alerts are logged.

FIG. 4B—STRUCTURED REVIEW. FIG. 4B illustrates review of bias indicators and equity signals. Review requires authorization. Outcomes are documented.

FIG. 4C—MITIGATION RECOMMENDATION. FIG. 4C depicts generation of non-directive mitigation recommendations. Recommendations may include retraining review. Recommendations do not enforce actions.

FIG. 4D—APPROVAL WORKFLOW. FIG. 4D illustrates approval workflows for mitigation actions. Approval requires human decision-making. Decisions are logged.

FIG. 4E—OUTCOME TRACKING. FIG. 4E depicts tracking of mitigation outcomes across monitoring windows. Tracking supports improvement. Records are immutable.

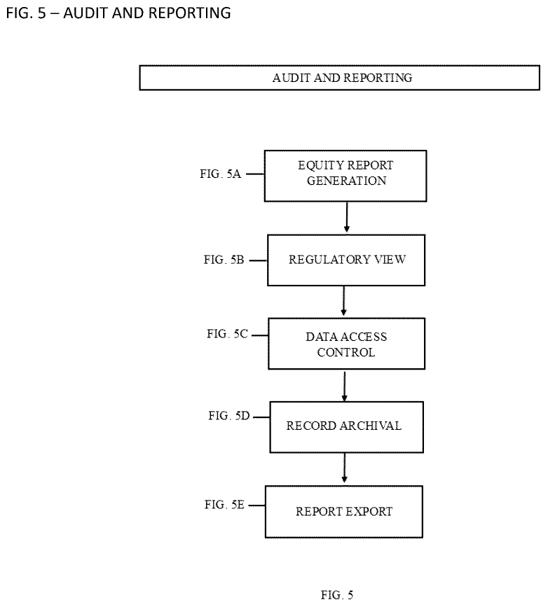

### FIG. 5—Audit and Reporting

FIG. 5 illustrates audit logging, reporting, and regulatory readiness. Records support institutional and external review. Reporting is standardized.

FIG. 5A—EQUITY REPORT GENERATION. FIG. 5A depicts generation of quality and equity reports. Reports are cohort-aware. Reports are versioned.

FIG. 5B—REGULATORY VIEW. FIG. 5B illustrates regulator-aligned reporting views. Views align with ethical AI frameworks. Views support audits.

FIG. 5C—DATA ACCESS CONTROL. FIG. 5C depicts role-based access control for governance data. Access events are logged. Controls are enforced.

FIG. 5D—RECORD ARCHIVAL. FIG. 5D illustrates archival of governance records. Records are retained per policy. Long-term oversight is supported.

FIG. 5E—REPORT EXPORT. FIG. 5E depicts export of reports for external review. Export formats are standardized. Patient identifiers are excluded.

### Illustrative Operational Example

In one example, an AI-assisted stroke triage workflow is monitored across multiple patient cohorts within a healthcare institution. Stratified response time and escalation metrics are computed across defined cohorts.

The system detects a statistically significant disparity in escalation latency and generates an equity signal. Governance stakeholders review the signal using the governance interface.

A mitigation action is documented and tracked over subsequent monitoring windows. The system does not autonomously diagnose conditions or prescribe treatment.

1. A computer-implemented system comprising one or more processors and memory storing instructions that cause the system to compute stratified quality metrics for AI-assisted clinical workflows, detect bias indicators and equity signals across defined cohorts, generate governance records, and produce auditable reports, wherein the system does not autonomously diagnose a medical condition or prescribe treatment.

2. A method comprising capturing workflow data associated with AI-assisted care, defining cohorts, computing stratified performance metrics, detecting equity signals, and generating governance records.

3. A non-transitory computer-readable medium storing instructions that cause one or more processors to perform the method of claim 2.

4. The system of claim 1, wherein equity signals are based on statistically significant disparities.

5. The system of claim 1, wherein performance drift is detected across monitoring windows.

6. The system of claim 1, wherein mitigation workflows require human approval.

7. The system of claim 1, wherein quality thresholds are configurable.

8. The system of claim 1, wherein governance records are immutable.

9. The method of claim 2, wherein cohort definitions are policy-driven.

10. The system of claim 1, wherein regulator-aligned reports are generated.
